(19) United States
(12) Patent Application Publication
(10) Pub. No.: US 2002/0115599 A1
(43) Pub. Date: Aug. 22, 2002

(54) MICROTUBULE-ASSOCIATED TPX2 PROTEIN

(76) Inventors: ISABLLE VERNOS, HEIDELBERG (DE); ERIC KARSENTI, HEIDELBERG (DE); TORSTEN WITTMANN, HEIDELBERG (DE)

Correspondence Address: ARENT FOX KINTNER PLOTKIN AND KAHN, PLLC, 1050 CONNECTICUT AVE. N.W., SUITE 600, WASHINGTON, DC, 20036-5339, US

(21) Appl. No.: 09/431,226

(22) Filed: Nov. 1, 1999

(30) Foreign Application Priority Data
Apr. 1, 1999 (EP) 99 106 710.9
May 31, 1999 (EP) 99 110 507.3

(51) Int. Cl. A61K 38/00; A61K 31/70; C07K 14/00; C07K 17/00; C07K 1/00

(52) U.S. Cl. 514/12; 530/350

(57) ABSTRACT

A novel microtubule-associated protein is decribed which is involved in mechanisms during mitosis.

The present invention relates to TPX2, a novel microtubule-associated protein which is involved in mechanisms during mitosis.

During mitosis, chromosomes are segregated by a complex microtubule-based structure, the mitotic spindle. The chromatides of each chromosome separate and migrate under the influence of the mitotic spindle to one of the two cell poles each.

Current models of spindle assembly involve the action of a variety of mictrotubule motors exerting forces on microtubules (Barton et al, Proc. Natl. Acad. Sci. USA 93 (1996), 1735-1742; Vernos et al, Curr. Opin. Cell Biol. 8 (1996), 4-9). Kinesin, for example, is a mechano-chemical enzyme acting as microtubule motor. Xklp2 is a plus end-directed Xenopus kinesin-like protein localized at spindle poles and required for centrosome separation during spindle assembly in Xenopus egg extracts (H. Boleti et al., Cell 84 (1996) 49-59; E. Karsenti et al., Sem. Dev. Cell. Biol., 7 (1996) 367-378).

To understand the functioning of motors in spindle morphogenesis, further investigations are necessary. In particular, different levels of their functions have to be examined. One level concerns the mechanisms of motor localization through specific interactions between the variable nonmotor domains and their cargoes (Afshar et al, Cell 81 (1995), 129-138). A second level relates to the actual motor function in relation to the activity of other plus and minus end-directed motors (Hoyt et al, J. Cell Biol. 118 (1992), 109-120). A third level concerns the regulation of the activity and localization of motors in time and space by posttranslational modifications (Blangy et al, Cell 83 (1995), 1159-1169).

Recently, the localization of the kinesin-like protein Xklp2 to spindle poles was reported as being dependent on the dimerization of Xklp2, a COOH-terminal leucine zipper, a microtubule-associated protein (MAP), and the activity of the dynein-dynactin complex (Wittmann et al, J. Cell Biol. 143 (3) (1 998), 673-685). A structure or the sequence of the involved microtubule-associated protein was, however, not revealed.

It was therefore an object of the present invention to provide a novel microtubule-associated protein which is involved in mitosis.

The invention comprises a nucleic acid coding for TPX2 comprising:

(a) the nucleotide sequence shown in SEQ ID NO:1, SEQ ID NO:4 or SEQ ID NO: 6,

(b) a sequence corresponding to the nucleotide sequence shown in SEQ ID NO:1, SEQ ID NO:4 or SEQ ID NO:6 within the degeneration of the genetic code,

(c) a sequence having a homology greater than 80% to the nucleotide sequence of SEQ ID NO:1, SEQ ID NO:4 or SEQ ID NO:6

(d) a sequence being a section of the nucleotide sequence of (a), (b) or/and (c) having at least 50 bases therefrom,

(e) a sequence which hybridizes with at least one of the sequences (a) to (d) under stringent conditions,

(f) a genomic sequence containing one of the sequences (a) to (e) and further containing one or more introns, or

(g) a sequence which differs from a sequence (a) to (f) due to its origin from a different species.

The term TPX2 is short for “targeting protein for Xklp2”. However, not only proteins from Xenopus are comprised by the term TPX2 as used herein but also corresponding proteins from other species, in particular proteins from mammals, preferably humans.

TPX2 functions by assisting the binding of kinesin-like proteins (KLP) having a motor function to microtubules during mitosis. TPX2 itself preferably binds directly to microtubules or/and kinesin-like proteins (KLP), in particular Xklp2 or human kinesin-like protein. Preferably, TPX2 is a receptor for a leucine zipper of the kinesin-like protein. Xklp2 for example was found to have a leucine zipper at the COOH-terminus.

In general TPX2 mediates the binding of kinesin-like proteins to microtubules independent of the species from which it is derived. In particular it mediates the binding of the COOH-terminal domain of kinesin-like proteins, preferably Xklp2, to microtubules. It was found that the protein encoded by SEQ ID NO:1 mediates the binding of Xklp2 as well as the binding of GST(glutathione-S-transferase)-Xklp2-tail to microtubules, indicating that the COOH-terminal domain of KLP is involved in the binding mechanism.

TPX2, however, need not be the only protein required for the localization of the kinesin-like protein on the microtubules, but other prtoteins may be involved as well. An example for such an other protein is the dynein-dynactin complex.

The nucleotide acid according to the invention preferably is a DNA. However, it may also be a cDNA, an RNA or a nucleic acid analog, such as a peptidic nucleic acid or a modified nucleic acid. Such modified nucleic acids may carry for example additional substituents on the nucleobases, the sugar moieties or the phosphate linking groups. Such substituents may alter the properties of the nucleic acid with regard to the desired application.

In one embodiment of the invention, the nucleic acid comprises a sequence having a homology of greater than 80%, preferably greater than 90%, and more preferably greater than 95% to the nucleotide sequences according to SEQ ID NO:1, SEQ ID NO:4 or SEQ ID NO:6 or to sequences corresponding thereto within the degeneration of the genetic code, while still encoding a protein having the described features.

While the nucleic acid according to the invention preferably comprises one of the sequences shown in SEQ ID NO:1, 4 or 6, it may also comprise only a portion therefrom, as long as the properties of the encoded protein are substantially retained. Such a portion comprises a section being at leat 50, preferably at least 100, more prefarably at least 200, and most preferably at least 500 nucleobases of one of the nucleotide sequences (a), (b) or/and (c) described above. A section comprising the coding sequence (CDS) for the protein is particularly preferred.

The term “hybridization under stringent conditions” according to the present invention is used as described by Sambrook et al (Molecular Cloning, A Laboratory Manual, Cold Spring Harbor Laboratory Press (1989), 1.101-1.104). Preferably, a stringent hybridization according to the present invention is given when after washing for an hour with 1×SSC and 0.1% SDS at 50° C., preferably at 55° C., more preferably at 62° C., and most preferably at 68° C., and more preferably for one hour with 0.2×SSC and 0.1% SDS at 50° C., preferably at 55° C., more preferably at 62° C., and most preferably at 68° C. a positive hybridization signal is still observed. A nucleotide sequence which hybridizes under such washing conditions with any of the sequences of (a) to (d), preferably with the nucleotide sequence shown in SEQ ID NO:1, SEQ ID NO:4 or SEQ ID NO:6, is a nucleotide sequence according to the invention.

The nucleic acid preferably contains the sequence as of SEQ ID NO:1 coding for Xenopus laevis eggs TPX2, the sequence as of SEQ ID NO:3 being the cDNA of Xenopus laevis, the sequence as of SEQ ID NO:4 coding for part of the human TPX2 or the sequence as of SEQ ID NO:6 coding for the human TPX2.

SEQ ID NO:4 or sections therefrom can be used by the skilled artisan to obtain the complete cDNA and genomic DNA of human TPX2 by standard protocols known in the art, e.g. as hybridization probes for screening suitable libraries. The complete open reading frame for human TPX2 is shown in SEQ ID NO:6.

The nucleic acids according to the invention can be obtained using known techniques, e.g. using short sections of the nucleotide sequences disclosed herein as hybridization probe or/and primer. They can, however, also be produced by chemical synthesis.

The nucleic acid according to the present invention preferably is an operative association with an expression control sequence. The expression control sequence is preferably active in eukaryotic cells, most preferably in Xenopus or in mammal cells.

The invention further comprises a recombinant vector containing at least one copy of a nucleic acid according to the invention. This may be a prokaryotic or a eukaryotic vector which contains the nucleic acid according to the invention under the control of an expression signal, in particular a promoter, operator, enhancer etc. Examples of prokaryotic vectors are chromosomal vectors such as bacteriophages and extra-chromosomal vectors such as plasmids, circulary plasmid vectors being particularly preferred. Prokaryotic vectors useful according to the present invention are described in, e.g., Sambrook et al, supra. Such prokaryotic vectors can be used to introduce the nucleic acid of interest into a eukaryotic cell.

More preferably, the vector according to the invention is a eukaryotic vector, in particular a vector for mammal cells, preferably a vector for human cells. Most preferred are vectors suitable for gene therapy, such as retrovirus, modified adenovirus or adeno-associated virus. Such vectors are known to the man skilled in the art and are also described, e.g., in Sambrook et al, supra.

The invention further comprises a cell transformed with a nucleic acid or a vector according to the invention. The cell may be a eukaryotic or prokaryotic cell, eukaryotic cells being preferred. The cell may be used as bioreactor to produce the protein of interest.

The invention also comprises a polypeptide encoded by a nucleic acid according to the invention, in particular a polypeptide comprising SEQ ID NO:2, SEQ ID NO:5 or SEQ ID NO:7. It also relates to polypeptides differing therefrom by additions, substitutions, deletions or/and insertions of amino acids that do not substantially affect its activity. The modifications preferably concern single amino acids or short amino acid sections having less than 10, preferably less than 5 amino acids.

The polypeptide is, e.g., obtainable by expression of the corresponding nucleic acid sequence in a suitable expression system (cf. Sambrook et al, supra). The polypeptide can also be fused to other polypeptides or/and proteins to build a fusion protein.

The polypeptide according to the invention, or fragments thereof, can be used for the production of an inhibitor of the respective polypeptide. In particular it can be used as immunogen for the production of antibodies, whereby standard protocols for obtaining antibodies may be used. Since KLP function is essential for mitotic spindle assembly, such inhibitors, preferably antibodies, which interfere with TPX2 are useful to target specifically mitotic cells, and in particular for the treatment of cancer.

Further, the present invention also encompasses a pharmaceutical composition comprising as active component a nucleic acid, vector, polypeptide or inhibitor as described herein. The pharmaceutical composition may additionally comprise pharmaceutically acceptable carriers, vehicles and/or additives and additional active components, if desired. Such a pharmaceutical composition can be used for diagnostic or/and therapeutic purposes. Particularly preferred is the use for affecting mitotic cells and, preferably, for the treatment of cancer.

Since TPX2 is involved in the localization of kinesin-like protein to the microtubules during mitosis, which KLP function in turn is essential for centrosome separation during prophase and the assembly of a bipolar spindle, mitosis can be affected by the addition of TPX2 or its antagonist, respectively. An effect on mitosis can take place on both the nuleic acid and the polypeptide level.

Therefore a use of the nucleic acid according to the invention is in the production of an inhibitor of said nucleic acid. Such an inhibitor can be produced by techniques known to the man skilled in the art. The inhibitor interferes with the activity of the nucleic acid and can be used to inhibit or prevent mitosis of cells, which is of particular interest in cells showing abnormally enhanced mitosis.

On the other hand, the nucleic acid according to the invention can be used directly to induce or/and promote mitosis or to compensate for a cell deficiency.

The active component on nucleic acid level may be formulated into a pharmaceutical composition by techniques known in the art, e.g. using liposomes as carrier or using suitable vectors, e.g. herpes vectors.

More preferably, a polypeptide having TPX2 activity or its antagonist, respectively, are used for affecting mitosis rather than a nucleic acid. Again, a polypeptide having TPX2 activity can be used for inducing and/or promoting mitosis or for the compensation of cell deficiencies.

An inhibitor, preferably an antibody, which interferes with TPX2 activity, can be used to inhibit or/and prevent mitosis, which is particularly useful to target cancer cells.

Since TPX2 is involved in mitosis through the binding of Xklp2-tail to microtubules, this gene and protein, respectively, can also be used to design drug target structures.

The invention is further illustrated by the appending sequences and figures wherein

SEQ ID NO:1 shows the DNA sequence for Xenopus laevis TPX2;

SEQ ID NO:2 shows the Xenopus laevis TPX2 protein sequence (715 amino acids);

SEQ ID NO:3 shows the Xenopus laevis TPX2 cDNA sequence (3539 kb);

SEQ ID NO:4 shows part of the coding sequence for human TPX2;

SEQ ID NO:5 shows the partial protein sequence of human TPX2;

SEQ ID NO:6 shows the open reading frame of the human TPX2 and

SEQ ID NO:7 shows the protein sequence of the human TPX2.

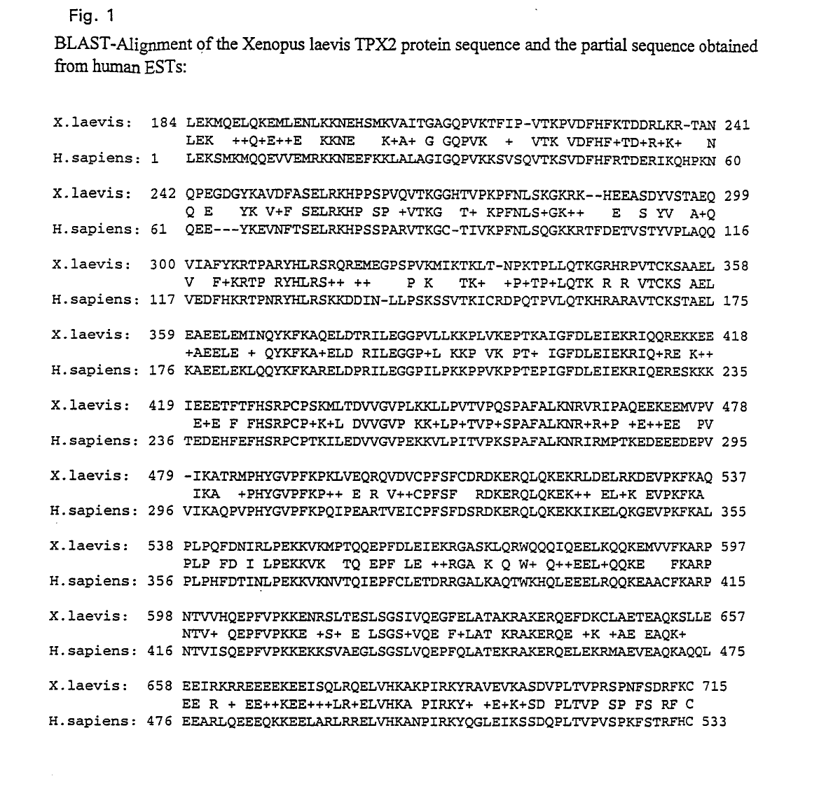

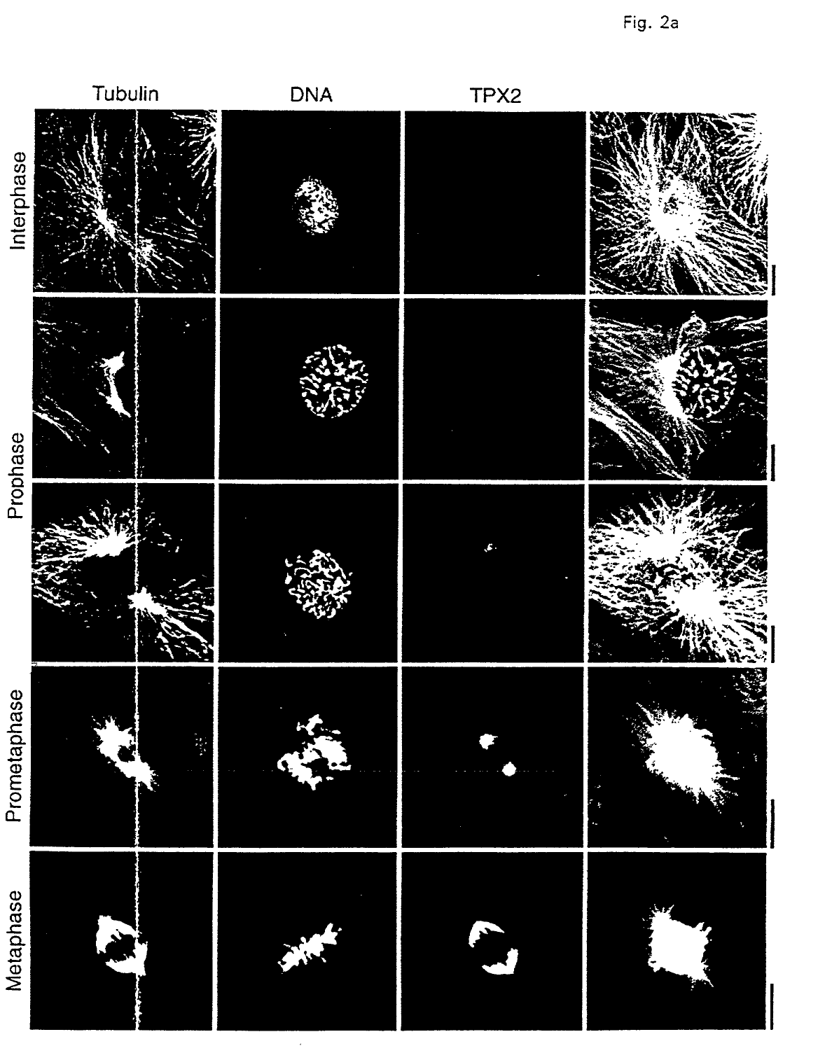

FIG. 1 shows a BLAST-alignment of the Xenopus laevis TPX2 protein sequence and the partial sequence obtained from human ESTs.

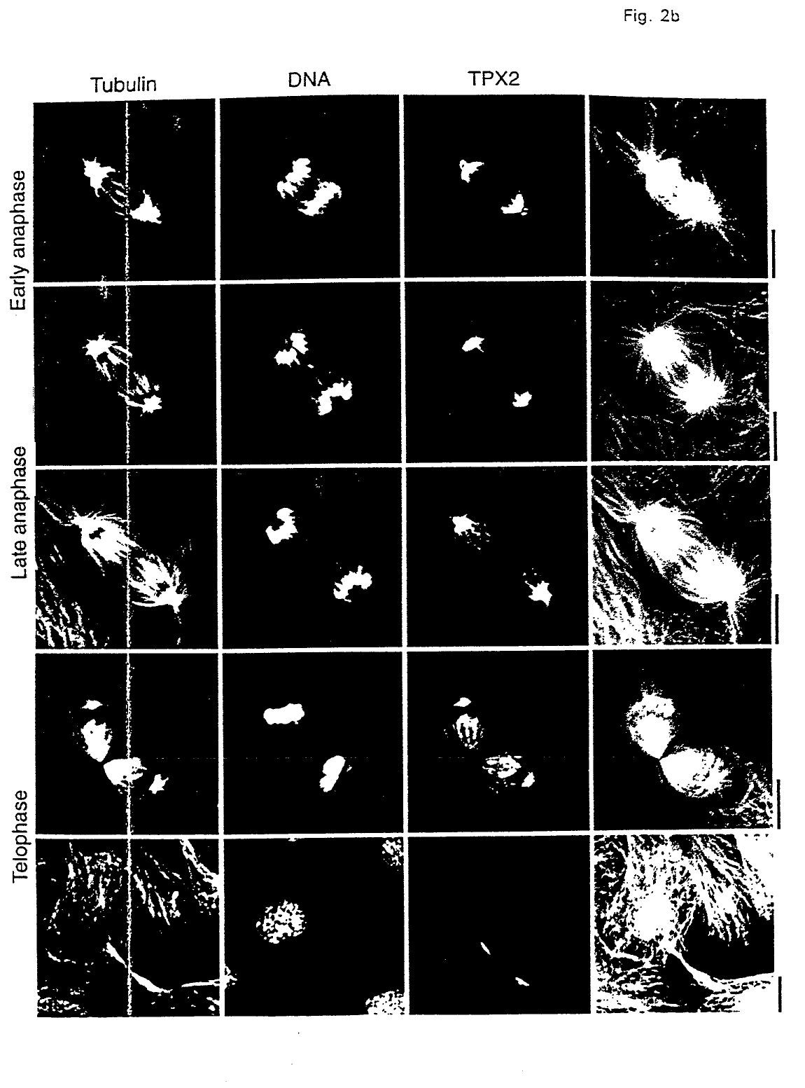

FIG. 2 (two pages): shows localization of TPX2 throughout the cell cycle. Methanol-fixed XL177 cells were stained with an affinity-purified anti-TPX2 antibody (#5843, 3^{rd} bleed, used at a concentration of 0.6 μg/ml), a monoclonal anti-tubulin antibody and Hoechst No. 33258. FITC-labeled anti-mouse and TRITC-labeled anti-rabbit antibodies were used as secondary antibodies. Images represent single confocal slices. The same staining pattern as with the #5837 serum is observed. TPX2-staining appears at the end of prophase, persists at the spindle poles throughout mitosis until late anaphase. It then relocalizes to the midbody where it disappears in late telophase. Bars, 10 μm.

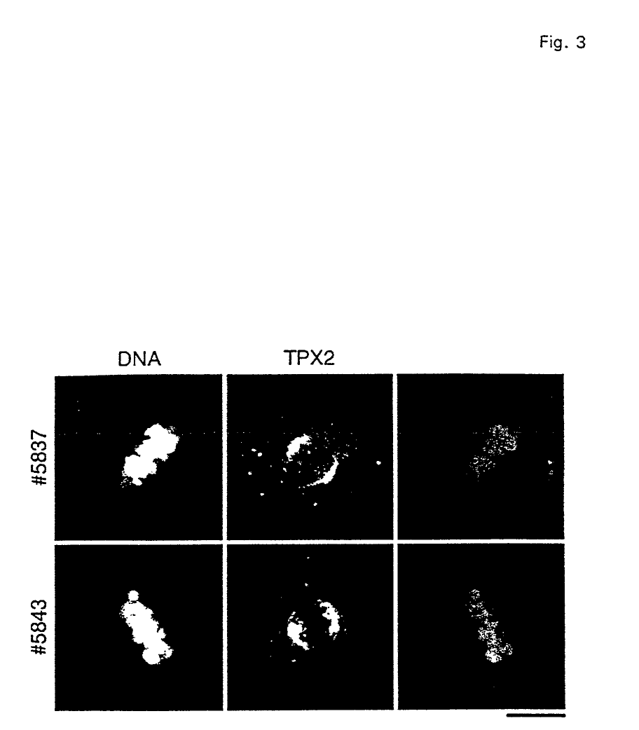

FIG. 3 shows HeLa cells in metaphase. The cells were fixed in cold methanol and stained with Hoechst No. 33258 and both affinity purified anti-TPX2 antibodies (at a concentration of 2-3 μg/ml). Both antibodies strongly stain the mitotic spindle. However, the cytoplasmic background is higher than in XL177 cells probably due to the higher antibody concentration that was necessary. Bar, 10 μm.

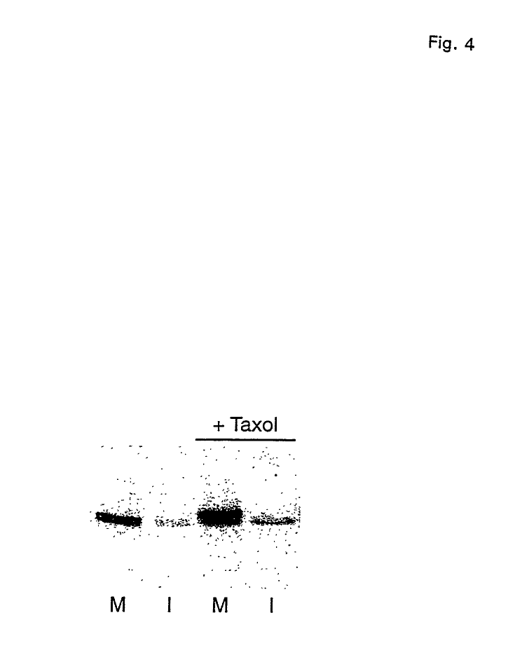

FIG. 4 shows phosphorylation of endogenous TPX2 in mitotic, M, and interphase, I, egg extract incubated in the presence of [γ-³²P]ATP. Taxol was added to 2 μM concentration and the extracts were incubated for 30 min at 20° C. The immunoprecipitates were analyzed on a 6% SDS-PAGE and subjected to autoradiography. TPX2 is phosphorylated in mitotic extract and hyperphosphorylated upon assembly of microtubules.

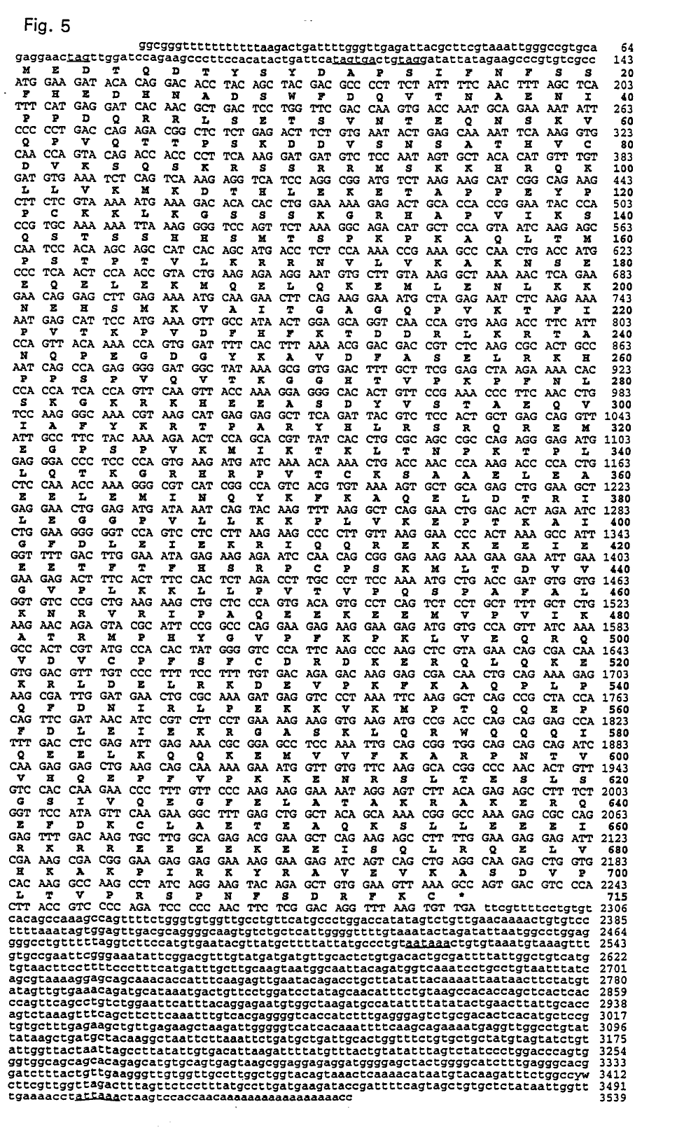

FIG. 5 shows the DNA sequence for Xenopus laevis TPX2 and the Xenopus laevis TPX2 protein sequence.

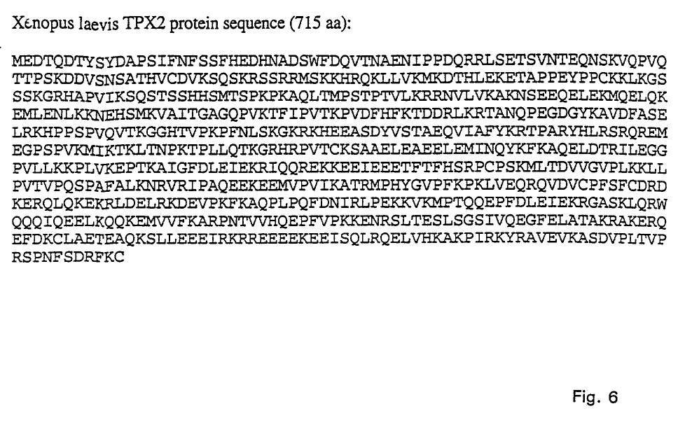

FIG. 6 shows the Xenopus laevis TPX2 protein sequence (715 aa).

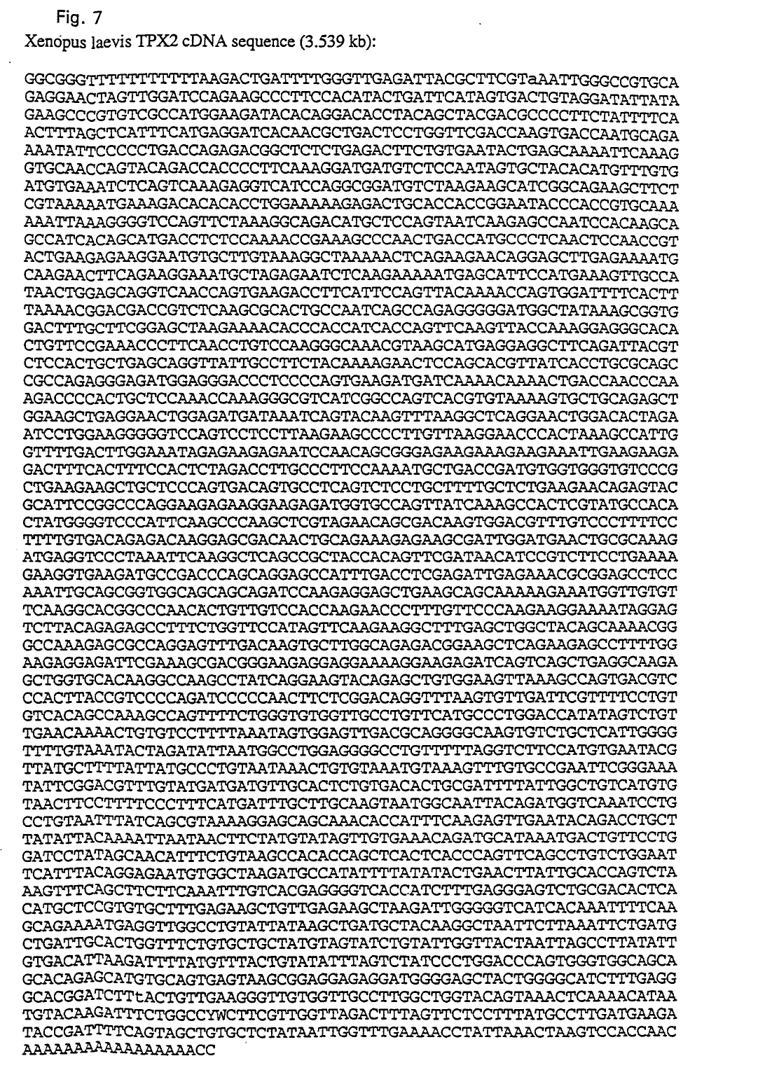

FIG. 7 shows the Xenopus laevis TPX2 cDNA sequence (3.539 kb).

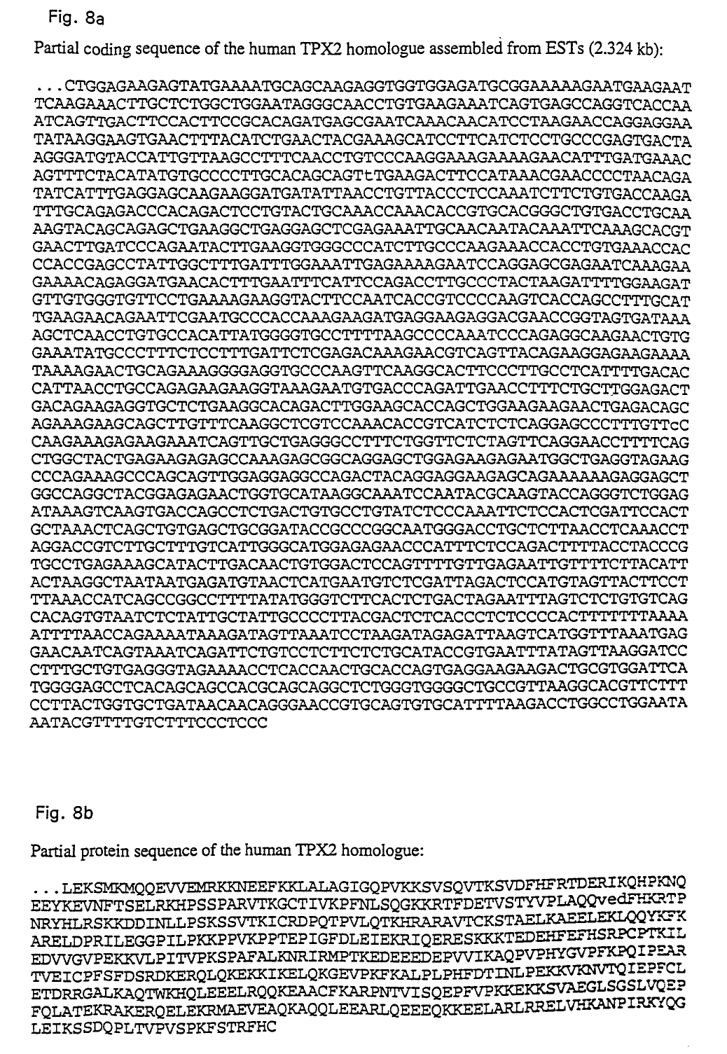

FIG. 8a shows the partial coding sequence of the human TPX2 homologue assembled from ESTs (2.324 kb).

FIG. 8b shows the partial protein sequence of the human TPX2 homologue.

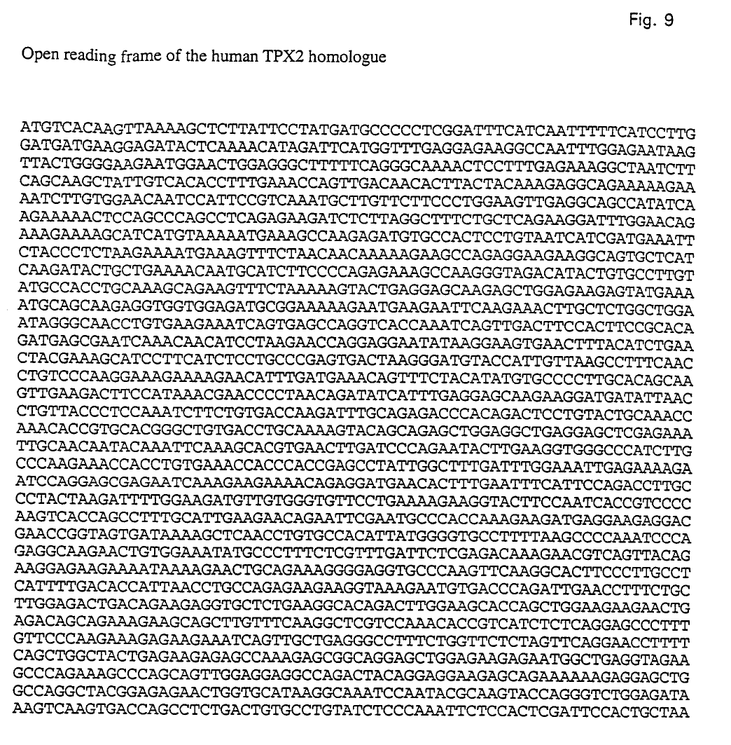

FIG. 9 shows the human TPX2 DNA-sequence.

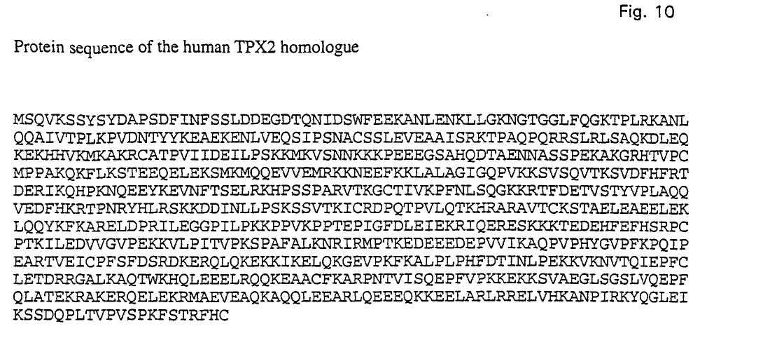

FIG. 10 shows the protein sequence of the human TPX2 homologue.

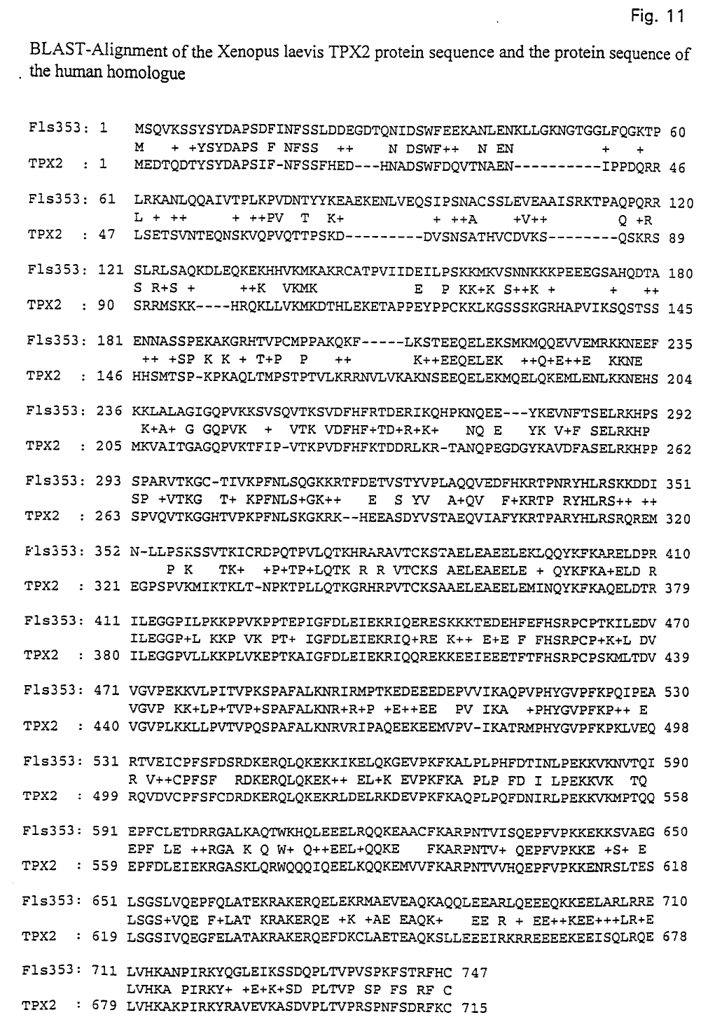

FIG. 11 shows a BLAST-Alignment of the Xenopus laevis TPX2 protein sequence and the protein sequence of the human homologue.

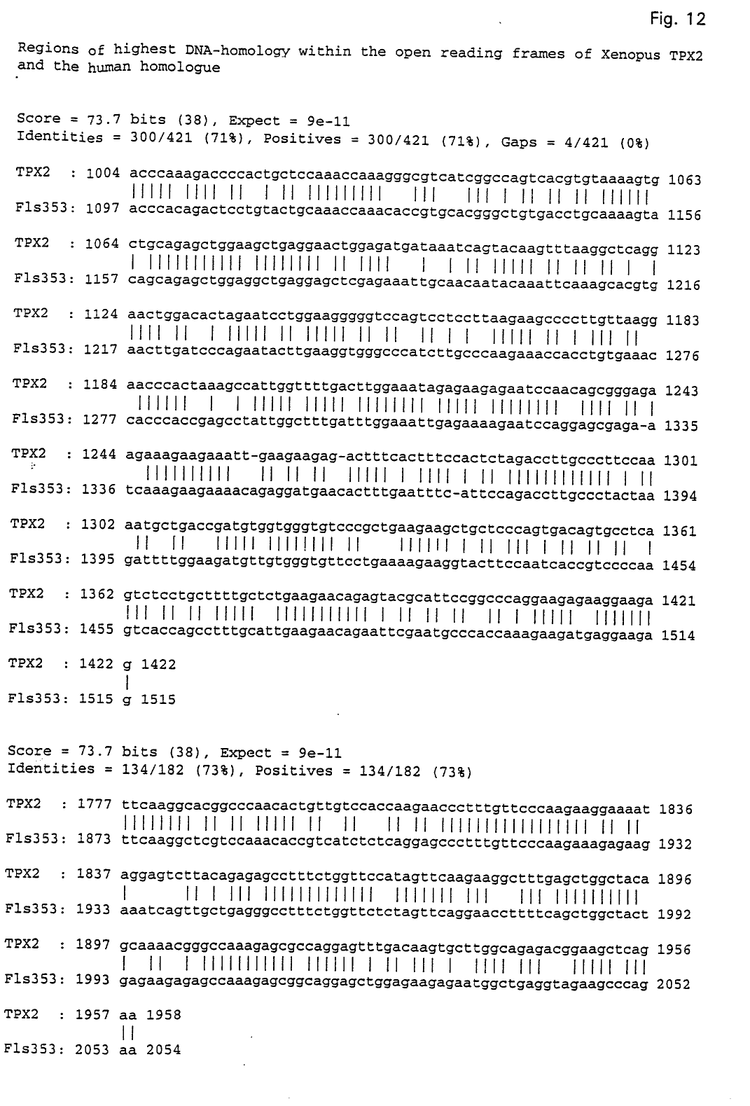

FIG. 12 shows regions of highest DNA-homology within the open reading frames of Xenopus TPX2 and the human homologue.

### EXAMPLES

### Example 1: Purification of TPX2

1.1 Xenopus Egg Extracts

CSF-arrested extracts (mitotic extracts) were prepared according to Murray, Cell Cycle Extracts, in Methods in Cell Biology, Vol. 36, B. K. Kay and H. B. Peng, editors, Academic Press, San Diego, Calif. (1991), 581-605. They were released to interphase by addition of 0.5 mM CaCl₂ and 200 μg/ml cycloheximide and subsequent incubation at 20° C. for 45-60 min. High speed extracts were centrifuged for 60 min at 150,000 g at 4° C.

1.2 Purification

To determine the additional protein which is required for GST-Xklp2-tail (a glutathione-S-transferase fusion protein containing the COOH-terminal domain of Xklp2 (Boleti et al., Cell 84 (1996) 49-59)) binding to microtubules MAPs were prepared from CSF-arrested egg extract. CSF-arrested egg extract was diluted with two volumes of motor buffer (100 mM K-PIPES, pH 7.0, 0.5 mM EGTA, 2.5 mM magnesium acetate, and 1 mM DTT) containing 10 μg/ml pepstatin, leupeptin, aprotinin, and 1 mM PMSF, and then centrifuged for 90 min at 180,000 g at 4° C. The cytoplasmic layer was collected and supplemented with 0.6 mg/ml bovine brain tubulin (Ashford et al, Preparation of tubulin from bovine brain, in Cell Biology: A Laboratory Handbook, vol.2, J. E. Celis, editor, Academic Press, San Diego, Calif. (1998), 205-212) and 20 μM taxol and microtubules were polymerized at room temperature for 30 min. The microtubules were then centrifuged through a 15% sucrose cushion in motor buffer (30,000 g, 20 min, 22° C. ) containing 5 μM taxol. The supernatant was discarded, the cushion once washed with water, removed and the microtubule pellet resuspended in ⅓ of the original volume in motor buffer and taxol, and then centrifuged through a sucrose cushion again. GST-Xklp2-tail alone does not bind to pure taxol-stabilized microtubules. However, a fraction of MAPs contains an activity that mediates the binding of GST-Xklp2-tail to microtubules. Only in the presence of the MAP-fraction a substantial amount of GST-Xklp2-tail was recovered in the microtubule pellet. It was observed by immunofluorescence that in this case GST-Xklp2-tail bound all along the prepolymerized microtubules. These results indicate the presence of a factor in mitotic egg extracts required for the binding of the COOH-terminal domain of Xklp2 microtubules that was enriched in a MAP fraction prepared from CSF-arrested egg extract.

1.3 Further Purification

As a further purification step, mitotic MAPs were eluted from microtubules in 100 mM steps of NaCl in motor buffer for 15 min at room temperature. Between the elution steps microtubules were recovered by centrifugation (30,000 g, 15 min, 22° C.). TPX2 was enriched in the fraction eluted with 300 mM NaCl.

1.4 Mono S Column

The fraction was diluted to reduce the salt concentration and applied onto a PC 1.6/5 Mono S column (SMART system; Pharmacia Biotech Sverige). The column was eluted with a 1-ml linear gradient of 100-500 mM KCl in 20 mM K-PIPES, pH 7.0, 10% glycerol, 1 mM EDTA, 1 mM DTT, 0.01% Tween-20 at 4° C. at 25 μl/min and 50-μl fractions were collected. TPX2 eluted at about 350 mM KCl in a single peak, corresponding to a doublet of polypeptide with molecular masses of about 100 kD. The strong affinity of these proteins for the Mono S suggested that they are highly basic.

1.5 Superdex 200 Gel Filtration Column

Since the Mono S peak fraction still contained some minor contaminants, it was further purified on a Superdex 200 gel filtration column. The peak fraction from the Mono S chromatography was applied to a PC 3.2/30 Superdex 200 gel filtration column (SMART system; Pharmacia Biotech Sverige) equilibrated with 20 mM K-Hepes, pH 7.0, 300 mM KCl, 10% glycerol, 1 mM EDTA, 1 mM DTT, and 0.01% Tween-20 at 4° C. The column was eluted with the same buffer at a flow rate of 20 μl/min and 40-μl fractions were collected. The column fractions were assayed in the following way: 4 mg/ml cycled bovine brain tubulin was polymerized in BRB80 containing 5 mM MgCl₂, 33% glycerol and 1 mM GTP at 37° C. for 30 min. The microtubules were stabilized by the addition of 20 μM taxol. 5 μl MAPs or column fractions were mixed with 10 μl BRB80 containing 1 mM DTT, 5 μM taxol, 0.1% Triton X-100 and 1 μM GST-Xklp2-tail. 5 μl of the prepolymerized microtubules were added and the reactions incubated at 20° C. for 30 min. The reactions were then diluted 1:5 with BRB80 containing 1 mM DTT, 5 μM taxol, and 0.1% Triton X-100, and then centrifuged through a 10% sucrose cushion in BRB80, 5 μM taxol at 200,000 g at 20° C. for 15 min. The cushion was washed once with water and the microtubule pellet solubilized in SDS-PAGE sample buffer and analyzed by Western blotting with an anti-GST antibody.

The anti-GST antibody was affinity purified against GST (glutathion-S-transferase) from a rabbit serum immunized with an unrelated GST-fusion protein.

Obtained was a MAP which is named TPX2 herein (targeting protein for Xklp2) and mediates the binding of the COOH-terminal domain of Xklp2 to microtubules. Purifed TPX2 itself is capable of rebinding to pure microtubules. There is a high probability that TPX2 is the receptor for the leucine zipper found at the COOH-terminus of Xklp2.

### Example 2: Cloning of TPX2

Sequencing of the purified protein TPX2 obtained in Example 1 was performed. Subsequently, a polymerase chain reaction (PCR) with degenerate oligos was conducted, followed by screening of a Xenopus cDNA library.

2.1 Obtaining TPX2 Peptide Sequence

TPX2 was purified from mitotic egg extract as described in Example 1. The peak fractions after Mono S chromatography of several preparations from about 60-70 ml of crude extract altogether were pooled and run on a preparative protein gel. After Coomassie-staining, the 100-kD band corresponding to TPX2 was cut out and the protein digested with trypsin. The tryptic peptides were then subjected to peptide sequencing by tandem electrospray mass spectroscopy.

Several peptide sequences were obtained by mass spectroscopy that proved to be useful for designing primers (Table 1). Mass spectroscopic sequencing can not distinguish between leucine and isoleucine because of their identical mass leading to an ambiguity in these positions. Furthermore, for two peptides two slightly divergent sequences were obtained either reflecting sequencing errors or different isoforms present in the purified TPX2 fraction. Database searches with these peptide sequences did not reveal any similarities to known proteins.

TABLE 1

| | |
|---|---|
| Peptide sequence | Degenerate oligonucleotides |

| | |
|---|---|
| . . . (V)TVPQSPAFA(L/I)K | |
| (L/I)(L/I)PVTVPQSPAFPSK | A5: CCI GTN ACI GTN CCI CAR |
| | WSI CCN GC |
| | A3: GC NGG ISW YTG IGG IAC |
| | NGT IAC NGG |
| . . . GFD(L/I)E(L/I)EQR | B5: GGI TTY GAY HTI GAR HTN |
| | GAR C |
| | 83: TG YTC IAD YTC IAD RTC |
| | RAA NCC |
| ILEGGPVLLK† | |
| ILEGGPVLPK† | C5: TN GAR GGI GGN CCI GTI |
| | YTN CC |
| | 03: YTT IGG NAR IAC NGG ICC |
| | NCC YTC |
| AVDFASEIR† | D5: GCN GTI GAY TTY GCI WSN |
| | GAR |
| | D3: YTC ISW NGC RAA RTC IAC |
| | NGC |
| (TQ)PVDFGVQK | E5: CCN GTI GAY TTY GGN GTN |
| | C |
| | E3: TG NAC ICC RAA RTC NAC |
| | NGG |
| (L/I)(L/I)EYF(L/I)(L/I)K | |

Table 1: TPX2 peptide sequences obtained by tandem electrospray mass spectroscopy and the derived degenerate oligonucleotides used for PCR. The peptide sequences indicated in bold were used for primer design. Brackets indicate uncertainties. (↑) These peptide sequences were confirmed by Edman degradation and, therefore, are not ambigous at the leucine positions. Standard degenerate nucleotid alphabet: R(A/G), Y(C/T), K(G/T), M(A/C), S(G/C), W(A/T), B(G/C/T), D(A/GIT), H(A/C/T), V(A/C/G), N(any), and I(inosine).

2.2 RNA Isolation, cDNA Synthesis and Degenerate PCR

Since the order of the peptides in the TPX2 protein sequence was unknown, five pairs of degenerate oligonucleotides were designed pointing in both the upstream and downstream direction (Table 1) based on the least ambiguous parts of the peptide sequences. The primers were 19-26 nucleotides long and contained up to four inosines to keep the degeneracy low (256-576 fold) taking into account all possible permutations of the amino acid sequence. Inosine presumably base pairs equally well with all four nucleotides.

Poly(A)⁺-mRNA was isolated from approximately 1 mg of total Xenopus egg RNA using the PolyATract mRNA Isolation System (Promega) following the suppliers instructions. Briefly, the RNA is hybridized to a biotinylated oligo(dT) primer that is captured and washed at high stringency using streptavidin coupled paramagnetic beads. The poly(A)⁺-mRNA was precipitated in the presence of 625 mM ammonium acetate, 1 μg glycogen and 66% ethanol at −70° C. for 30 min.

After centrifugation and drying, the RNA pellet was dissolved in sterile water containing 25 ng oligo(dT)₂₂ heated to 70° C. for 10 min and chilled on ice. The reaction was then adjusted to 50 μl 50 mM Tris-HCl pH 8.3, 75 mM KCl, 3 mM MgCl₂, 10 mM DTT, 1 unit RNAse block (Stratagene) and 0.5 mM dNTPs and prewarmed to 42° C. 500 units SuperScript II reverse transcriptase (Gibco BRL) were added and cDNA was synthesized at 42° C. for 90 min. The reaction was terminated by incubation at 70° C. for 15 min. This mixture was directly used as a template for PCR.

The degenerate PCR was performed in 50 μl reactions containing 20 mM Tris-HCl pH 8.4, 50 mM KCl, 1.5 mM MgCl₂, 300 μM dNTPs, 5 units AmpliTaq (Perkin-Elmer), 1 μM of the degenerate primers in all possible combinations and 0.1 to 0.5 μl of the RNA/cDNA template using the following program:

| | | | |
|---|---|---|---|
| 94° C. | 2 | min | |
| 40° C. or 50° C. | 1 | min | |
| 72° C. | 1 | min | |
| 94° C. | 30 | sec | \| |
| 40° C. or 50° C. | 30 | sec | \| 30-35 cycles |
| 72° C. | 1 | min | \| |
| 72° C. | 5 | min | |

After polishing the ends with Pfu DNA polymerase, the PCR-fragments were subcloned with the pCR-Script Amp SK(+) cloning kit (Stratagene).

2.3 Library Screening

| | | | |
|---|---|---|---|
| 20x SSC, pH 7.0: | 3 | M | NaCl |
| | 0.3 | M | Trisodium citrate |

Library screening was done according to standard protocols (Ausubel et al., 1995. Short Protocols in Molecular Biology. John Wiley & Sons, Inc.). The PCR-fragments obtained with the primer pairs D5/C3 and C5/A3 were radiolabeled by random priming and DNA synthesis in the presence of [α³²P]dCTP (Amersham LifeScience) using High Prime (Boehringer Mannheim), purified on Sephadex G-50 gel filtration columns (NICK column from Pharmacia Biotech) and used to screen a mature Xenopus oocyte cDNA library in λ-ZAP (obtained from John Shuttleworth, University of Birmingham). The library contained the inserts in the EcoRI site of the pBluescript phagemid.

Phages were grown in top agar containing a lawn of Escherichia coli BB4 cells. Plaque lifts were taken with Hybond-N nylon filters (Amersham Life Science) for 30-60 seconds, denatured in 0.5 M NaOH, 1.5 M NaCl for 1 min, neutralized in 0.5 M Tris-HCl pH 8.0, 1.5 M NaCl for 5 min and finally accumulated in 2×SSC. The DNA was cross-linked to the filters using an UV Stratalinker 2400 (Stratagene).

The filters were prehybridized in 50% deionized formamide, 1 M NaCl, 1% SDS, 10% dextran sulphate and 100 μg/ml sheared denatured salmon sperm DNA for 2-3 hours at 42° C. Subsequently, the radiolabeled probe was added and hybridized at 42° C. overnight. The filters were washed twice in 2×SSC, 0.5% SDS, twice in 1×SSC, 0.5% SDS and once in 0.1×SSC, 0.1% SDS at 65° C. for 15 min each.

Positive plaques were cut out and eluted with approximately 0.5 ml 50 mM Tris-HCl pH 7.5, 100 mM NaCl, 8 mM MgSO₄, 0.01% gelatin containing a drop of chloroform for 1-2 hours at 4° C., diluted and plated for the next round of screening.

After three rounds of screening, positive pBluescript phagemids were isolated by in vivo excision using the ExAssist/SOLR system (Stratagene) following the suppliers instructions.

To fully sequence TPX2 clone #8 that produced the strongest band in the an vitro transcripiton/translation reaction was selected and progressive unidirectional deletions were prepared by digestion with exonuclease III using the Erase-a-Base System (Promega). The plasmid was either cut with KpnI and Sal I or with SacI and NotI to generate a nuclease-resistant and a nuclease-sensitive end close to the T7 or the T3 promoter, respectively. The exonuclease III reaction was done according to the suppliers instructions taking timepoints every 30-40 seconds. Sequencing was done with standard T7 or T3 primers.

Clone #8 contained one long open reading frame of 2145 bp starting at position 203. Probably the first ATG in the sequence also represented the start codon of the open reading frame since it was preceded by several in frame stop codons and the surrounding nucleotides matched reasonably well the consensus for eukaryotic translation initiation (GCC(A/G)CCATGG).

2.4 Analysis of the TPX2 Predicted Amino Acid Sequence

The open reading frame of clone #8 encoded a polypeptide of 715 amino acids with a predicted size of 82.4 kD. The predicted protein is highly charged (18.7% strongly basic and 14.7% strongly acidic amino acids) and extraordinarily basic (the predicted isoelectric point is about 9.5) possibly explaining its strong interaction with the cation exchanger resin during purification.

Apart from the last peptide shown in Table 1 that was not very specific in the first place, all other peptides obtained by mass spectroscopy could be identified in the predicted protein sequence. Minor differences may be due to peptide sequencing errors or the existence of different isoforms of the protein. Most of the differences observed between clone #8 and the peptide sequences can be explained by single nucleotide changes.

| | Homology | | | |
|---|---|---|---|---|
| Accession | in | | | |
| number | TPX2 | Identities | Similarities | Expect |
| AA828703 | amino acid | 60% | 72% | 7e⁻³⁶ |
| | 424-547 | | | |
| AA218615 | amino acid | 62% | 70% | 9e⁻³⁵ |
| | 424-517 | | | |
| AA134490 | amino acid | 59% | 73% | 5e⁻²⁸ |
| | 199-317 | | | |
| AA159064 | amino acid | 56% | 72% | 4e⁻²⁰ |
| | 226-348 | | | |
| AA136254 | amino acid | 53% | 60% | 2e⁻¹⁸ |
| | 594-686 | | | |

Table 2: Homology of human ESTs to the TPX2 protein sequence. The five most interesting out of 24 significant BLAST hits are listed (from December 1998). The query sequence was filtered for regions of low complexity.

Database searches with either the nucleotide or the predicted protein sequence did not reveal any obvious similarity to any protein characterized so far. Since TPX2 presumably is a microtubule-binding protein it was specifically compared to other MAPs. It showed no significant homology to NuMA and it was not homologous to the better defined microtubule binding domains of the the tau-family shared by tau, MAP2, MAP4 and chTOG, the human homolog of XMAP215 (Charrasse et al., 1998. The TOGp protein is a new human microtubule-associated protein homologous to the Xenopus XMAP215. J. Cell Sci. 111:1371-1383.; Drewes et al., 1998. MAPs, MARKs and microtubule dynamics. Trends Biochem. Sci. 23:307-311.) or of the CLIP-170/p150^{Glued} type (Pierre et al., 1992. CLIP-170 links endocytic vesicles to microtubules. Cell. 70:887-900.; Waterman-Storer et al., 1995. The p150^{Glued} component of the dynactin complex binds to both microtubules and the actin-related protein centractin (Arp-1). Proc. Natl. Acad. Sci. USA. 92:1634-1638.).

However, a BLAST search of the EST database revealed a number of vertebrate (mainly mammalian) sequences that showed a very high homology to clone #8 (Table 2). No matches were found to bacterial, yeast or Caenorhabditis elegans sequences. Thus, clone #8 encoded a novel protein representing Xenopus TPX2.

The TPX2 protein was predicted to be mainly α-helical, but no obvious structural domains were identified apart from two short regions (amino acid 171-208 and 650-684) that showed a significant probability for coiled-coil formation.

A closer look revealed that TPX2 contained seven potential nuclear localization sites conforming either to a 4-residue pattern composed of four basic amino acids or of three basic amino acids and either a histidine or a proline, or to a 7-residue pattern starting with a proline and followed within 3 residues by a basic segment containing 3 basic residues out of 4. However, these nuclear localization sites may be coincidental considering the high density of basic residues in this protein. TPX2 did not contain a bipartite nuclear localization signal.

More interestingly, the TPX2 protein sequence also contained several potential phosphorylation sites. Three consensus phosphorylation sites for protein kinase A, (K/R)(K/R)XS, three sites for cdc2 kinase, (S/T)PX(K/R), and five potential MAP-kinase sites, PX(S/T)P, were identified. The TPX2 protein did not contain a MARK kinase site, KXGS, that has been identified in the microtubule-binding domain of tau-like proteins.

### Example 3: Human TPX Homologue

To compare the Xenopus TPX2 to the homologous protein of a different species, clones for three of the human ESTs were obtained from the IMAGE consortium and sequenced from both ends. It turned out that all three ESTs originated from the same gene starting at different positions but terminating at the same position, probably the poly(A)-tail. The EST-sequences could be assembled into a putative human TPX2 homologous sequence missing only about 180 amino acids at its NH₂-terminus. A BLAST alignment revealed that the proteins were 63% identical (77% similarity) in this region. Also the human protein has a very basic isoelectric point (about 9.4) and contains a predicted coiled-coil domain at the same position. Both proteins end with a peculiar cysteine as last amino acid. Interestingly, one of the cdc2 sites and all four MAP-kinase sites present in this part of the protein are conserved between frog and human. This suggests that TPX2 function might be regulated by phosphorylation. The nuclear localization sites are less well conserved. The human homologue contains only two putative nuclear localization sites in this region and only one is conserved between frog and human.

### Example 4: Features and Properties of TPX2

The protein obtained in Example 2 binds to microtubules and to XkIp2 leucine zipper at the C-terminal domain. During mitosis, the protein TPX2 is localized at spindle poles. It is involved in the localization of Xklp2, a plus end-directed kinesin-like protein, to microtubule minus ends during mitosis. XkIp2 function is essential for centrosome separation during prophase and assembly of a bipolar mitotic spindle. Since Xklp2 function is essential for mitotic spindle assembly, TPX2 is useful for finding drugs interfering therewith by impairing the binding of TPX2 to microtubule minus ends or to Xklp2. Such drugs are useful to target specifically mitotic cells, in particular cancer cells.

4.1 Subcellular Localization of TPX2

Polyclonal antibodies were raised against a full length GST-TPX2 fusion protein (rabbit #5837) and against a truncated GST-fusion protein (rabbit #5843). Both sera were affinity purified on a column of untagged TPX2 covalently coupled to sepharose. On Western blots both antibodies recognized the recombinant untagged TPX2 as well as a single band of the same size in Xenopus egg extracts and in an SDS-lysate of XL177 Xenopus tissue culture cells. Interestingly, in mitotic egg extract and in a preparation of mitotic MAPs the band was upshifted fitting better to the apparent molecular weight of TPX2 that was observed in the original purification from mitotic egg extract. The upshift suggested a posttranslational modification, possibly phosphorylation, of TPX2 in mitosis.

Both antibodies were used for immunofluorescence. XL177 cells were grown on coverslips in 70% Leibovitz L-15 (Sigma Chemical Co.) containing 10% FCS. The coverslips were briefly rinsed with 70% PBS and fixed in methanol at −20° C. for 5-10 min and unspecific binding was blocked by incubation in PBS containing 2% BSA, 0.1% Triton X-100 for 10 min. Primary and secondary antibodies were diluted in the same buffer and added to the coverslips for 20-30 min. The coverslips were washed 3-4 times with IF buffer after each antibody incubation and at the end embedded in mowiol.

The affinity-purified antibodies yielded a cleaner signal, but also the crude rabbit sera worked well and showed the same staining pattern. XL177 cells grown on coverslips simply fixed in cold methanol without preextraction gave the best results. The staining pattern of glutaraldehyde fixed cells looked similar, but the cytoplasmic background was drastically increased. In interphase cells no prominent staining was observed. In particular, there was no labeling of microtubules or centrosomes. In some cells, the nuclei appeared to be stained above background, but this was not very clear. Also in prophase, when the chromatin started to condense, no particular staining pattern was observed. At some point, possibly around the time of nuclear envelope breakdown, TPX2 staining became apparent at the center of the asters and in prometaphase a bright staining was evident at the center of both asters. The labeling intensity still increased and at metaphase the spindle microtubules were brightly stained. The signal appeared to be more intense towards the spindle poles. Interestingly, astral microtubules were basically not stained. During anaphase, the staining at the spindle poles remained and in early anaphase no labeling was visible in the center of the spindle between the separating sister chromatids. Later, a relocalization to the midzone occured that resulted in a fairly strong staining of the midbody, the cones of overlapping microtubules that remain between the two dividing cells, in late telophase at the end of mitosis, leaving no staining behind in the centrosomal area (FIG. 2).

Sequencing of the human ESTs revealed a high degree of homology to TPX2. Therefore, it was also attempted to stain HeLa cells with the two anti-TPX2 antibodies. The antibody concentration had to be raised and the signal to noise ratio was drastically decreased, but it was still possible to observe a staining pattern similar to the one in Xenopus cells from prometaphase to anaphase. Especially the mitotic spindle in metaphase was heavily stained (FIG. 3).

4.2 Phosphorylation in Egg Extract

| | | | |
|---|---|---|---|
| Stop Buffer, pH 7.5: | 100 | mM | NaF |
| | 80 | mM | β-glycerophosphate |
| | 20 | mM | sodium pyrophosphate, Na₄P₂O₇ |
| | 20 | mM | EDTA |
| | 10 | μg/ml | aprotinin, pepstatin, leupeptin |
| | 2 | μM | microcystin |

AffiPrep protein A beads (BioRad) were washed 2-3 times with PBS containing 0.1% Triton X-100 and 5-10 μg affinity-purified polyclonal antibody was bound to 10 μl packed beads overnight at 4° C. The next morning, the beads were washed twice with PBS containing 0.1% Triton X-100 and twice with Stop buffer and were kept resuspended in Stop buffer.

30-50 μCi [γ-³²P]ATP (Amersham LifeScience) were added to 50 μl mitotic or interphase Xenopus egg extract and incubated for 20-30 min at 20° C. The reaction was stopped by addition of 100-200 μl Stop buffer containing 10 μl of the antibody beads. The mixture was incubated for one hour on a rotating wheel at 4° C. The beads were washed twice with Stop buffer and twice with PBS containing 0.1% Triton X-100. At the end, the beads were resuspended in SDS-PAGE sample buffer and analyzed by SDS-PAGE and autoradiography.

The protein immunoprecipitated from mitotic extract showed a retardation in gel electrophoresis and an increased incorporation of radioactivity indicating that it was specifically phosphorylated in mitosis (FIG. 4). More interestingly, a second phosphorylation event was observed in mitotic extract when taxol was added to induce microtubule polymerization. This hyperphosphorylation only occurred in mitotic but not in interphase extract. When the gel was quantified with a phosphoimager the increase in incorporation of radioactivity was about 3-5-fold from interphase to mitotic extract and another 2-3-fold upon addition of taxol to mitotic extract.

1. A nucleic acid coding for TPX2 comprising:
(a) the nucleotide sequence shown in SEQ ID NO:1, SEQ ID NO:4 or SEQ ID NO:6
(b) a sequence corresponding to the nucleotide sequence shown in SEQ ID NO:1, SEQ ID NO:4 or SEQ ID NO:6 within the degeneration of the genetic code,
(c) a sequence having a homology greater than 80% to the nucleotide sequence of SEQ ID NO:1, SEQ ID NO:4 or SEQ ID NO:6
(d) a sequence being a section of the nucleotide sequence of (a), (b) or/and (c) having at least 50 bases therefrom,
(e) a sequence which hybridizes with at least one of the sequences (a) to (d) under stringent conditions,
(f) a genomic sequence containing one of the sequences (a) to (e) and further containing one ore more introns, or
(g) a sequence which differs from a sequence (a) to (f) due to its origin from a different species.

2. A nucleic acid according to claim 1, encoding a protein which is involved in mitosis.

3. A nucleic acid according to claim 1 or 2, encoding a protein which binds to microtubules.

4. A nucleic acid according to any of claims 1 to 3, encoding a protein which binds to kinesin-like proteins.

5. A nucleic acid according to any of claims 1 to 4, characterized in that it contains a cDNA according to SEQ ID NO:3.

6. A recombinant vector characterized in that it contains at least one copy of a nucleic acid according to claims 1 to 5.

7. A vector according to claim 6, characterized in that it is a eukaryotic vector.

8. A cell characterized in that it is transformed with a nucleic acid according to any of claims 1 to 5 or with a vector according to claim 6 or 7.

9. Use of a nucleic acid according to any of claims 1 to 5 for the production of an inhibitor of said nucleic acid.

10. An inhibitor of a nucleic acid according to any of claims 1 to 5.

11. A polypeptide encoded by a nucleic acid according to any of claims 1 to 5.

12. A polypeptide according to claim 11, comprising the sequence shown in SEQ ID NO:2.

13. A polypeptide according to claim 11, comprising the sequence shown in SEQ ID NO:5 or SEQ ID NO:7.

14. A polypeptide according to any of claims 11 to 13, characterized in that it binds to microtubules and/or kinesin-like protein.

15. A polypeptide according to any of claims 11 to 14, characterized in that it contains deletions, substitutions, insertions or/and additions of amino acids that do not substantially affect its activity.

16. A polypeptide according to any of claims 11 to 15, wherein a second protein is fused to build a fusion protein.

17. Use of a polypeptide according to any of claims 11 to 16, or of a fragment thereof for the production of a TPX2-inhibitor.

18. Use according to claim 17 as immunogen for the production of an antibody.

19. An inhibitor of a polypeptide according to any of claims 11 to 16.

20. An inhibitor according to claim 19, characterized in that it is an antibody against the polypeptide.

21. Use of a polypeptide according to any of claims 11 to 16 or of an inhibitor according to claim 17 or 18 for inducing or inhibiting mitosis.

22. A pharmaceutical composition comprising:
(a) a nucleic acid according to any of claims 1 to 5,
(b) a recombinant vector according to claim 6 or 7,
(c) an inhibitor according to claim 10,
(e) a polypeptide according to- any of claims 11 to 16, or/and
(f) an inhibitor according to claim 17 or 18.

23. Use of a pharmaceutical composition according to claim 22 for affecting mitotic cells.

24. Use of an inhibitor according to claim 10 or 17 to 18 for the treatment of cancer.


SEQUENCE LISTING (7 sequences)

SEQ ID NO: 1 - DNA, 3539 bp, Xenopus laevis
  CDS (144)..(2291)
ggcgggtttttttttttaagactgattttgggttgagattacgcttcgtaaattgggccgtgcagaggaactagttggatccagaagcccttccacatactgattcatagtgactgtaggatattatagaagcccgtgtcgccatggaagatacacaggacacctacagctacgacgccccttctattttcaactttagctcatttcatgaggatcacaacgctgactcctggttcgaccaagtgaccaatgcagaaaatattccccctgaccagagacggctctctgagacttctgtgaatactgagcaaaattcaaaggtgcaaccagtacagaccaccccttcaaaggatgatgtctccaatagtgctacacatgtttgtgatgtgaaatctcagtcaaagaggtcatccaggcggatgtctaagaagcatcggcagaagcttctcgtaaaaatgaaagacacacacctggaaaaagagactgcaccaccggaatacccaccgtgcaaaaaattaaaggggtccagttctaaaggcagacatgctccagtaatcaagagccaatccacaagcagccatcacagcatgacctctccaaaaccgaaagcccaactgaccatgccctcaactccaaccgtactgaagagaaggaatgtgcttgtaaaggctaaaaactcagaagaacaggagcttgagaaaatgcaagaacttcagaaggaaatgctagagaatctcaagaaaaatgagcattccatgaaagttgccataactggagcaggtcaaccagtgaagaccttcattccagttacaaaaccagtggattttcactttaaaacggacgaccgtctcaagcgcactgccaatcagccagagggggatggctataaagcggtggactttgcttcggagctaagaaaacacccaccatcaccagttcaagttaccaaaggagggcacactgttccgaaacccttcaacctgtccaagggcaaacgtaagcatgaggaggcttcagattacgtctccactgctgagcaggttattgccttctacaaaagaactccagcacgttatcacctgcgcagccgccagagggagatggagggaccctccccagtgaagatgatcaaaacaaaactgaccaacccaaagaccccactgctccaaaccaaagggcgtcatcggccagtcacgtgtaaaagtgctgcagagctggaagctgaggaactggagatgataaatcagtacaagtttaaggctcaggaactggacactagaatcctggaagggggtccagtcctccttaagaagccccttgttaaggaacccactaaagccattggttttgacttggaaatagagaagagaatccaacagcgggagaagaaagaagaaattgaagaagagactttcactttccactctagaccttgcccttccaaaatgctgaccgatgtggtgggtgtcccgctgaagaagctgctcccagtgacagtgcctcagtctcctgcttttgctctgaagaacagagtacgcattccggcccaggaagagaaggaagagatggtgccagttatcaaagccactcgtatgccacactatggggtcccattcaagcccaagctcgtagaacagcgacaagtggacgtttgtcccttttccttttgtgacagagacaaggagcgacaactgcagaaagagaagcgattggatgaactgcgcaaagatgaggtccctaaattcaaggctcagccgctaccacagttcgataacatccgtcttcctgaaaagaaggtgaagatgccgacccagcaggagccatttgacctcgagattgagaaacgcggagcctccaaattgcagcggtggcagcagcagatccaagaggagctgaagcagcaaaaagaaatggttgtgttcaaggcacggcccaacactgttgtccaccaagaaccctttgttcccaagaaggaaaataggagtcttacagagagcctttctggttccatagttcaagaaggctttgagctggctacagcaaaacgggccaaagagcgccaggagtttgacaagtgcttggcagagacggaagctcagaagagccttttggaagaggagattcgaaagcgacgggaagaggaggaaaaggaagagatcagtcagctgaggcaagagctggtgcacaaggccaagcctatcaggaagtacagagctgtggaagttaaagccagtgacgtcccacttaccgtccccagatcccccaacttctcggacaggtttaagtgttgattcgttttcctgtgtcacagccaaagccagttttctgggtgtggttgcctgttcatgccctggaccatatagtctgttgaacaaaactgtgtccttttaaatagtggagttgacgcaggggcaagtgtctgctcattggggttttgtaaatactagatattaatggcctggaggggcctgtttttaggtcttccatgtgaatacgttatgcttttattatgccctgtaataaactgtgtaaatgtaaagtttgtgccgaattcgggaaatattcggacgtttgtatgatgatgttgcactctgtgacactgcgattttattggctgtcatgtgtaacttccttttccctttcatgatttgcttgcaagtaatggcaattacagatggtcaaatcctgcctgtaatttatcagcgtaaaaggagcagcaaacaccatttcaagagttgaatacagacctgcttatattacaaaattaataacttctatgtatagttgtgaaacagatgcataaatgactgttcctggatcctatagcaacatttctgtaagccacaccagctcactcacccagttcagcctgtctggaattcatttacaggagaatgtggctaagatgccatattttatatactgaacttattgcaccagtctaaagtttcagcttcttcaaatttgtcacgaggggtcaccatctttgagggagtctgcgacactcacatgctccgtgtgctttgagaagctgttgagaagctaagattgggggtcatcacaaattttcaagcagaaaatgaggttggcctgtattataagctgatgctacaaggctaattcttaaattctgatgctgattgcactggtttctgtgctgctatgtagtatctgtattggttactaattagccttatattgtgacattaagattttatgtttactgtatatttagtctatccctggacccagtgggtggcagcagcacagagcatgtgcagtgagtaagcggaggagaggatggggagctactggggcatctttgagggcacggatctttactgttgaagggttgtggttgccttggctggtacagtaaactcaaaacataatgtacaagatttctggccywcttcgttggttagactttagttctcctttatgccttgatgaagataccgattttcagtagctgtgctctataattggtttgaaaacctattaaactaagtccaccaacaaaaaaaaaaaaaaaaacc

SEQ ID NO: 2 - protein, 715 aa, Xenopus laevis
MEDTQDTYSYDAPSIFNFSSFHEDHNADSWFDQVTNAENIPPDQRRLSETSVNTEQNSKVQPVQTTPSKDDVSNSATHVCDVKSQSKRSSRRMSKKHRQKLLVKMKDTHLEKETAPPEYPPCKKLKGSSSKGRHAPVIKSQSTSSHHSMTSPKPKAQLTMPSTPTVLKRRNVLVKAKNSEEQELEKMQELQKEMLENLKKNEHSMKVAITGAGQPVKTFIPVTKPVDFHFKTDDRLKRTANQPEGDGYKAVDFASELRKHPPSPVQVTKGGHTVPKPFNLSKGKRKHEEASDYVSTAEQVIAFYKRTPARYHLRSRQREMEGPSPVKMIKTKLTNPKTPLLQTKGRHRPVTCKSAAELEAEELEMINQYKFKAQELEELEMINQPVLLKKPLVKEPTKAIGFDLEIEKRIQQREKKEEIEEETFTFHSRPCPSKMLTDVVGVPLKKLLPVTVPQSPAFALKNRVRIPAQEEKEEMVPVIKATRMPHYGVPFKPKLVEQRQVDVCPFSFCDRDKERQLQKEKRLDELRKDEVPKFKAQPLPQFDNIRLPEKKVKMPTQQEPFDLEIEKRGASKLQRWQQQIQEELKQQKEMVVFKARPNTVVHQEPFVPKKENRSLTESLSGSIVQEGFELATAKRAKERQEFDKCLAETEAQKSLLEEEIRKRREEEEKEEISQLRQELVHKAKPIRKYRAVEVKASDVPLTVPRSPNFSDRFKC

SEQ ID NO: 3 - DNA, 3539 bp, Xenopus laevis; identical to SEQ ID NO: 1

SEQ ID NO: 4 - DNA, 2324 bp, Homo sapiens
ctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcagtttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctgaaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcctttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgccccttacgactctcaccctctccccactttttttaaaaattttaaccagaaaataaagatagttaaatcctaagatagagattaagtcatggtttaaatgaggaacaatcagtaaatcagattctgtcctcttctctgcataccgtgaatttatagttaaggatccctttgctgtgagggtagaaaacctcaccaactgcaccagtgaggaagaagactgcgtggattcatggggagcctcacagcagccacgcagcaggctctgggtggggctgccgttaaggcacgttctttccttactggtgctgataacaacagggaaccgtgcagtgtgcattttaagacctggcctggaataaatacgttttgtctttccctccc

SEQ ID NO: 5 - protein, 533 aa, Homo sapiens
LEKSMKMQQEVVEMRKKNEEFKKLALAGIGQPVKKSVSQVTKSVDFHFRTDERIKQHPKNQEEYKEVNFTSELRKHPSSPARVTKGCTIVKPFNLSQGKKRTFDETVSTYVPLAQQVEDFHKRTPNRYHLRSKKDDINLLPSKSSVTKICRDPQTPVLQTKHRARAVTCKSTAELKAEELEKLQQYKFKARELDPRILEGGPILPKKPPVKPPTEPIGFDLEIEKRIQERESKKKTEDEHFEFHSRPCPTKILEDVVGVPEKKVLPITVPKSPAFALKNRIRMPTKEDEEEDEPVVIKAQPVPHYGVPFKPQIPEARTVEICPFSFDSRDKERQLQKEKKIKELQKGEVPKFKALPLPHFDTINLPEKKVKNVTQIEPFCLETDRRGALKAQTWKHQLEEELRQQKEAACFKARPNTVISQEPFVPKKEKKSVAEGLSGSLVQEPFQLATEKRAKERQELEKRMAEVEAQKAQQLEEARLQEEEQKKEELARLRRELVHKANPIRKYQGLEIKSSDQPLTVPVSPKFSTRFHC

SEQ ID NO: 6 - DNA, 2244 bp, Homo sapiens
  CDS (1)..(2244)
atgtcacaagttaaaagctcttattcctatgatgccccctcggatttcatcaatttttcatccttggatgatgaaggagatactcaaaacatagattcatggtttgaggagaaggccaatttggagaataagttactggggaagaatggaactggagggctttttcagggcaaaactcctttgagaaaggctaatcttcagcaagctattgtcacacctttgaaaccagttgacaacacttactacaaagaggcagaaaaagaaaatcttgtggaacaatccattccgtcaaatgcttgttcttccctggaagttgaggcagccatatcaagaaaaactccagcccagcctcagagaagatctcttaggctttctgctcagaaggatttggaacagaaagaaaagcatcatgtaaaaatgaaagccaagagatgtgccactcctgtaatcatcgatgaaattctaccctctaagaaaatgaaagtttctaacaacaaaaagaagccagaggaagaaggcagtgctcatcaagatactgctgaaaacaatgcatcttccccagagaaagccaagggtagacatactgtgccttgtatgccacctgcaaagcagaagtttctaaaaagtactgaggagcaagagctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcaagttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcgtttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaa

SEQ ID NO: 7 - protein, 747 aa, Homo sapiens [sequence omitted]